(19) United States
(12) Patent Application Publication
(10) Pub. No.: US 2010/0318009 A1
(43) Pub. Date: Dec. 16, 2010

(54) Therapeutic device that provides stimulation to an immobilized extremity

(76) Inventor: Solomon Tony Stanley, Visalla, CA (US)

Correspondence Address: ROBERT Z. EVORA, 4741 PLANTERS WALK, DOUGLASVILLE, GA, 30135, US

(21) Appl. No.: 12/806,654

(22) Filed: Aug. 17, 2010

Related U.S. Application Data
(63) Continuation-in-part of application No. 11/346,073, filed on Feb. 2, 2006, now abandoned.
(60) Provisional application No. 60/751,357, filed on Dec. 16, 2005.

(51) Int. Cl. A61F 5/01; A61F 7/00; A61N 1/36

(52) U.S. Cl. 602/14; 602/5; 602/23; 607/3; 602/21

(57) ABSTRACT

A therapeutic device for biasing a body portion of a patient into a predetermined position. The therapeutic device includes a rigid support that is substantially flat and contoured to brace the body portion into the predetermined position. A securing mechanism is provided to brace the rigid support against the body portion and a therapeutic stimulation mechanism is provided. The therapeutic stimulation mechanism can include an electrical stimulation mechanism or a heating mechanism to provide heating and electrical stimulation to the body portion.

### CROSS REFERENCE TO RELATED APPLICATIONS

This application is a Continuation in Part Application which claims the benefit of the filing date of U.S. Non-Provisional application Ser. No. 11/346,073, entitled “THERAPEUTIC DEVICE THAT PROVIDES STIMULATION TO AN IMMOBILIZED EXTREMITY” filed Feb. 2, 2006, the entirety of which is incorporated herein by reference; which claims the benefit of the filing date of U.S. Provisional Application Ser. No. 60/751,357, entitled “A THERAPEUTIC DEVICE THAT PROVIDES STIMULATION TO AN IMMOBILIZED EXTREMITY” filed Dec. 16, 2005, the entirety of which is incorporated herein by reference.

### BACKGROUND

1. Field of the Invention

The present invention relates in general to a rigid therapeutic device for immobilized portions of a person's body who suffer from a lack of muscular control and/or may be unconscious, and in particular to providing therapeutic stimulation with heat and/or electrical pulses to the severely deformed extremity.

2. Description of the Related Art

Loss of muscular control is oftentimes afflicted to individuals who have suffered from a stroke, comatose patients, palsy patients, arthritis patients, and those who have had tendon surgery. In many cases, loss of muscular control causes the patient's hands to clench shut and their feet to curl downward into a clenched configuration. When forced open, there is a tendency for their hands and feet to bias back into the unsightly stressed position. In order to reposition the hand or foot into a preferred unclenched position, the extremity must be urged back into the desired configuration. In other instances, stimulation may be provided to the damaged extremity in addition to therapy to reorient the extremity into a preferred operable position.

Attempts have been made to construct splints which allow some residual mobility. However, conventional devices fail to secure a patient's extremity in a preferred position as well as provide therapy to the damaged extremity simultaneously.

### SUMMARY OF THE INVENTION

An object of the present invention is to provide a therapeutic device for immobilized portions of a patient's body who may be unconscious and who has suffered a severe injury that has rendered a lack of muscular control in their extremities.

Another object of the present invention is to provide therapeutic stimulation to the patient's immobilized extremity by providing heat and/or electric impulses as the stimulus.

According to this invention, a therapeutic device is provided for biasing a body portion of a patient into a predetermined position. A substantially flat rigid support that is contoured to the patient's hand is provided to brace the body portion into the predetermined position. A securing mechanism braces the rigid support against the body portion and a therapeutic stimulation mechanism is included. The therapeutic stimulation mechanism includes an electrical stimulation mechanism and a heating mechanism to provide heating and electrical stimulation to the body portion at desired amounts and/or desired intervals.

The therapeutic stimulation mechanism is programmable and adapted to provide predetermined amounts of heating and electrical simulation to the body portion.

A further object of the present invention is to integrate the therapeutic stimulus in a glove to a patient's immobilized hand or in a booty (or sock-like covering) to the patient's immobilized foot.

Still another object of the present invention is to provide therapeutic device having a rigid contoured padded portion secured to the patient's hand or foot that serves as a splint to prevent the hand or foot from biasing into a clenched closed configuration. In addition, the therapeutic device also includes therapeutic stimulation to the patient's immobilized extremity by providing heat and/or electric impulses as the stimulus.

These and other objects, features, and/or advantages may accrue from various aspects of embodiments of the present invention, as described in more detail below.

### BRIEF DESCRIPTION OF THE DRAWINGS

Various exemplary embodiments of this invention will be described in detail, wherein like reference numerals refer to identical or similar components or steps, with reference to the following figures, wherein:

FIG. 1 is an illustration of a contoured pad on a therapeutic device for a hand.

FIG. 2 is a side view illustration of the patient's hand fastened to the contoured pad on a therapeutic device.

FIG. 3 is an illustration of a contoured pad on a therapeutic device for a foot.

FIG. 4 is a side view illustration of the patient's foot fastened to the contoured pad on a therapeutic device.

FIG. 5 is an illustration of a therapeutic device configured as a pair of gloves having a stimulation mechanism including a therapeutic heating control mechanism.

FIG. 6 is a side view illustration of the therapeutic device secured to a contoured pad including the therapeutic heating control mechanism.

FIG. 7 is an illustration of a therapeutic device having another stimulation mechanism including an electrical mechanism that provides a therapeutic electro-massage to stimulate the patient's hand.

FIG. 8 is an illustration of a pair of therapeutic devices having a stimulation mechanism comprised of a pair of electrical mechanisms that provide a therapeutic electro-massage to stimulate the patient's hand.

FIG. 9 is an illustration of the stimulation mechanism disposed in a contoured pad.

FIG. 10 is an illustration of a stimulation mechanism disposed in the cushion disposed on a contoured pad.

FIG. 11 is an illustration of a therapeutic device configured as a sock-like booty having a stimulation mechanism comprised of a heating control mechanism.

FIG. 12 is an illustration of a therapeutic device configured as a sock-like booty attached to the contoured pad and including the stimulation mechanism.

FIG. 13 is a front side illustration of a therapeutic device configured as a glove having a therapeutic stimulation mechanism with a heating control mechanism and an electric mechanism.

FIG. 14 is a bottom side illustration of a therapeutic device configured as a glove having a therapeutic stimulation mechanism with a heating control mechanism and an electric mechanism.

FIG. 15 is a side view illustration of a therapeutic device configured as a glove having a therapeutic stimulation mechanism with a heating control mechanism and an electric mechanism.

FIG. 16 is a first side illustration of a contoured pad for a therapeutic device adapted for use with a therapeutic heating control mechanism disposed in the straps above and below the hand.

FIG. 17 is a front view illustration of a patient's hand fastened to the contoured pad on the therapeutic device receiving therapeutic heat stimulation above and below the hand.

FIG. 18 is a second side view of the contoured pad for a therapeutic device adapted for use with the therapeutic heating control mechanism disposed above and below the hand.

FIG. 19 is a first side illustration of a contoured pad for a therapeutic device adapted for use with a therapeutic heating control mechanism disposed in the straps above and below a foot.

FIG. 20 is a front view illustration of a patient's foot fastened to the contoured pad on the therapeutic device receiving therapeutic heat stimulation above and below the foot.

FIG. 21 is a second side view of the contoured pad for a therapeutic device adapted for use with the therapeutic heating control mechanism disposed above and below the foot.

FIG. 22 is a first side illustration of a contoured pad including a therapeutic device adapted for use with a therapeutic electro-massage control mechanism disposed in the straps above and below the hand.

FIG. 23 is a front view illustration of a patient's hand fastened to the contoured pad on the therapeutic device receiving therapeutic electro-massage stimulation above and below the hand.

FIG. 24 is a second side view of the contoured pad for a therapeutic device adapted for use with the therapeutic electro-massage mechanism disposed above and below the hand.

FIG. 25 is a first side view of a contoured pad including a therapeutic device adapted for use with a therapeutic electro-massage mechanism disposed in the straps above and below a foot.

FIG. 26 is a front view illustration of a patient's foot fastened to the contoured pad on the therapeutic device receiving therapeutic electro-massage stimulation above and below the foot.

FIG. 27 is a second side view of the contoured pad for a therapeutic device adapted for use with the therapeutic electro-massage mechanism disposed above and below the foot.

FIG. 28 is another exemplary showing of a therapeutic device secured to a contoured pad including a therapeutic heating control mechanism providing heat therapy to both side of a hand.

FIG. 29 is yet another exemplary showing of a therapeutic device secured to a contoured pad providing therapeutic electro-massage therapy to both sides of a hand.

FIGS. 30-31 provide a pictorial exemplary illustration of the benefits of acupressure applied to the feet and hands of a person.

### DETAILED DESCRIPTION OF EXEMPLARY EMBODIMENTS

Particular embodiments of the present invention will now be described in greater detail with reference to the figures.

This invention overcomes the conventional problems described above by providing a therapeutic device for an immobilized portion of an extremity of a person. Deterioration of muscular control, which oftentimes shows up as a severe deformity in a patients hand or foot, and immobilization of an extremity occurs in a variety of different ways.

Loss of muscular control is oftentimes afflicted to individuals who have suffered from a stroke, comatose patients, palsy patients, arthritis patients, and/or those who have had tendon surgery. In many cases, loss of muscular control causes the patient's hands to clench shut and their feet to curl downward into an unnaturally clenched configuration. For example, in the instance of a severe heart attack and/or drastic loss of oxygen to the brain, the body's unnatural reaction is for the muscles in the body to severely clench up in an unsightly unnatural manner. The prolonged lack of oxygen to the brain and other critical portions of the body may cause permanent damage to the muscular control that person has over the various parts of their body, such as their hands and feet.

For example, in many instances, the foot of a person who suffered from a severe accident in which a lack of oxygen caused a severe muscular deformity, the flat lower portion of their foot curls downward, like a claw, such that the toes curl downward toward the heel of the foot into an unsightly and unnaturally hyper-flexed clenched position. Similarly, the muscles in the hands and the wrist of the patient curl downward and back toward the elbows into an unsightly and unnaturally hyper-flexed clenched position.

Even when the deformed hands and/or feet are forced open and/or pried into what would have been a natural state before the traumatic event; there is a tendency for the hands and feet of the deformed patient to spring back into the hyper-flexed and unsightly distressed and unnaturally position. In order to reposition the hand or foot into a more normal preferred unclenched and un-deformed position, the extremity, such as the hand or foot, must be urged back with sufficient force to overcome the unnaturally contracted muscles into a desired natural looking configuration. Unfortunately, these patients who have suffered from the recent traumatic event are unconscious and unable to walk. Consequently, active therapy to the deteriorated muscle is not possible by the patient until they have awaken and/or become cognizant in order to participate in the therapy.

Although various rehabilitation attempts have been made to assist a patient who is able to place some degree of weight onto their hands and/or feet and with some minimal degree of mobility, no solution has ever been contemplated and/or provided for a patient who is unable to walk, and is unconscious and has suffered from severe traumatic muscular deterioration.

Conventional therapeutic devices are only adapted for person's who have some minimal degree of mobility and can place their own weight onto their extremities. However, no device before has addressed securing and rehabilitating a severely deformed unconscious patient's extremity into a preferred position. That is, the preferred position being one in which the body portion can be firmly braced from the unnaturally clenched body position completely back into a natural unclenched and functional position prior to the muscular deteriorating condition. Nor, has any other method or device contemplated providing therapy to the damaged extremity of an unconscious person who has suffered the traumatic muscular deterioration and who is unable to walk and consequently cannot wear a conventional walking shoe, sandal or the like.

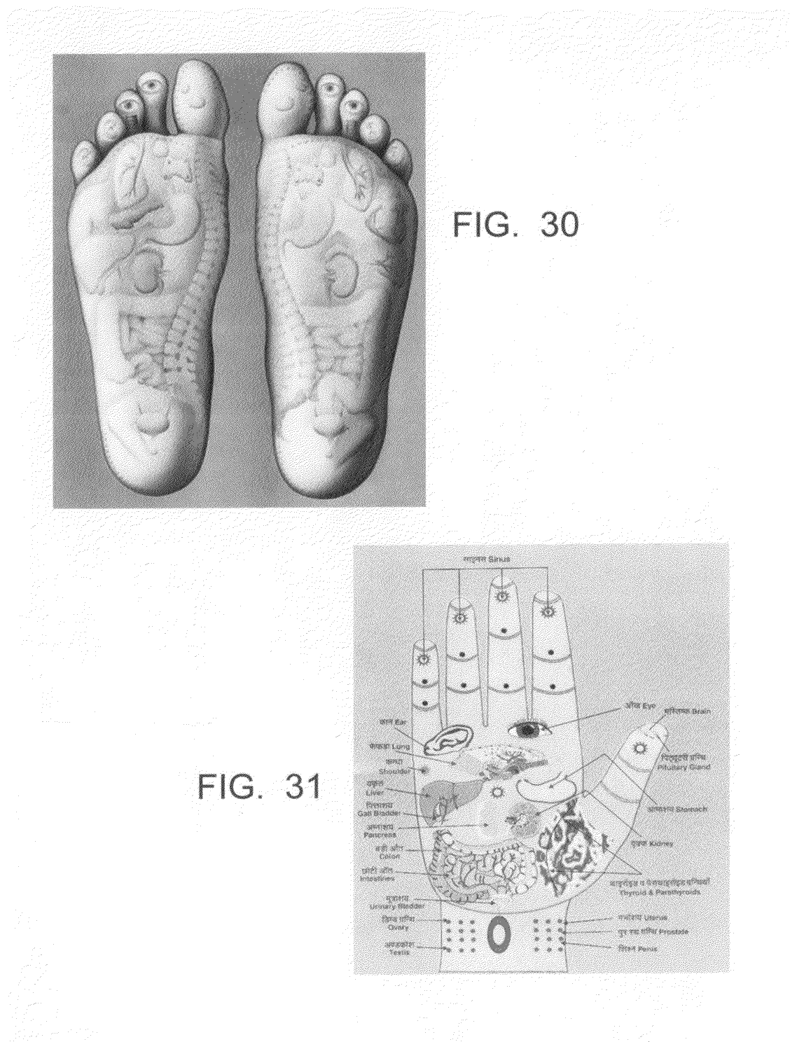

Understanding the well known benefits of acupressure applied to the feet and hands of a person as shown in FIGS. 30-31, this inventions leverages the benefits realized from applying various types of therapeutic stimulus to various parts of the hands and feet in order to facilitate the recover and rehabilitation process for an immobilized person by employing the use and understanding of pressure points throughout the body to stimulate healing, restore balance and energy flow in the body.

The cardiovascular benefits of acupressure in combination with the various embodiments disclosed herein may also be used to assist in reducing tension, improving circulation, lowering anxiety and increase relaxation, which all positively benefit a person's cardiovascular health.

Adapted for a Patient's Hand

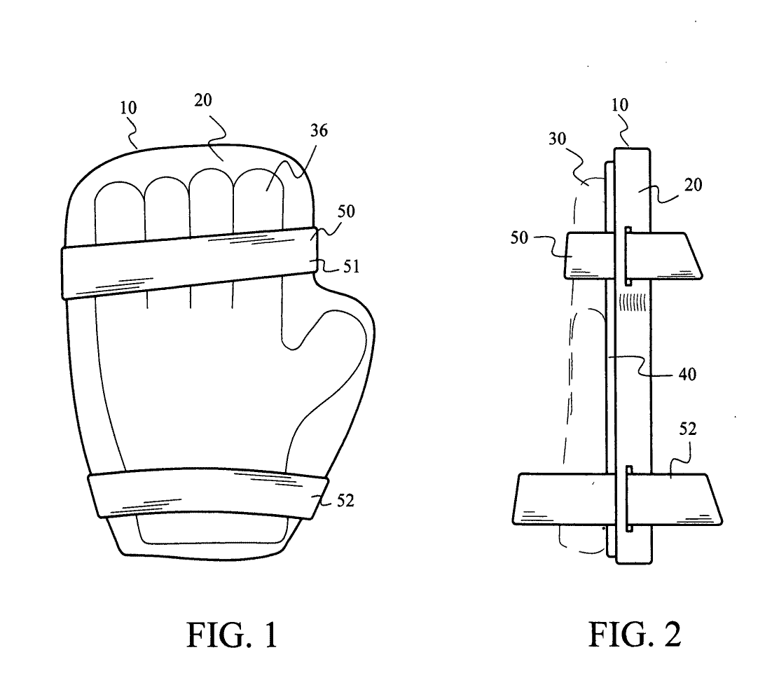

FIGS. 1 and 2 are exemplary illustrations of a therapeutic device 10 for a patient's hand 30. The therapeutic device 10 includes a contoured pad 20. The contoured pad 20 may be shaped along the outer silhouette of the hand 30 or any shape capable of restraining a portion of the patient's hand. The contoured pad 20 is made out of a rigid material to brace the hand 30.

The contoured pad 20 may include any number of solid materials including a polymer, a metal, and/or any other material suitable for providing a rigid structure. The rigidity of the contoured pad 20 is sufficiently strong enough to overcome a hyper-flexed deformed hand (or foot) as described above in the unnaturally deformed state in which the atrophy of the muscle may have set in and/or the patient is unconscious (and consequently cannot voluntarily place any of his weight onto the had or foot) and/or unwilling to assist in overcoming the severe deformity.

The surface of the contoured pad 20 that the hand 30 lies against may be supported by a flat soft cushion 40 composed of a material such as, felt, cotton, sponge, rubber and or any other soft substance now known or later developed. The cushion 40 may be disposed on the contoured pad 20 with an adhesive, by being sewn thereon, and/or any other method for fastening the cushion 40 to the contoured pad 20. The cushion 40 and/or the contoured pad 20, without the cushion 40, may include a depression 36 that the patient's hand (as shown in FIG. 1) can be secured onto to provide added comfort, stability and to accurately positioning of the fingers in a desired orientation.

Straps 50, 51, 52 are disposed on a surface of the contoured pad 20 to brace the hand 30 to the contoured pad 20. As shown in FIGS. 1-2, the two straps 51, 52 are attached to, and employed on, the surface of the contoured pad 20. As shown, the first strap 51 fastens the patient's fingers to the contoured pad 20. A second strap 52 is shown securing the patient's wrist to the contoured pad 20. The length, width and position of the straps 50, 51, 52 on the contoured pad 20 are disposed so that the extremities of the person who suffers from a severe muscular deformity (such as where the muscles in the hands and the wrist of the patient curl downward and back toward the elbows into an unsightly and unnaturally hyper-flexed clenched position) may be tightly secured to force the hands back into a natural unclenched position without any assistance by the user.

Reference to strap 50 is intended to be generic and to all of the various straps shown and described in all of the various exemplary embodiments, such as 51, 52, 53, 54, 55, and the like for the various therapeutic device's 10 shown for both the hand and foot.

The straps 50 may include any number of mechanisms for securing, such as a buckle, Velcro, clamps, a tie or the like. Any number of straps may be applied to the upper surface of the contoured pad 20 to force down and hold the patient's hand 30 in a desired position. For example, numerous straps can be provided to secure each of the fingers of a severely deformed hand individually in place. Alternatively, a single strap can be interwoven directly within the contoured pad 20, or the cushion 40 to create various loops to receive each of the fingers individually.

The contoured pad 20 may be used to bias the selected body portion, such as a hand, a finger, or a foot, to a desired secured position to aid in the healing and rehabilitation process, such as after a traumatic injury, surgery, or to assist a patient with at least partially deteriorated muscle control. This desired position may be a position which promotes proper mending of bones, healing of tendons or muscle tissue. Alternatively, it may be the desired at-rest natural position of the patient's body portion to overcome displacement caused by palsy, bone deformity, or other similar disablement such as a spasmed closed hand or foot.

In at least another aspect of the configuration of the therapeutic device 10, less emphasis is placed on the back side of the contoured pad 20 as it may be primarily used for patients who are unconscious and will not be applying any pressure onto any other object.

Adapted for a Patient's Foot

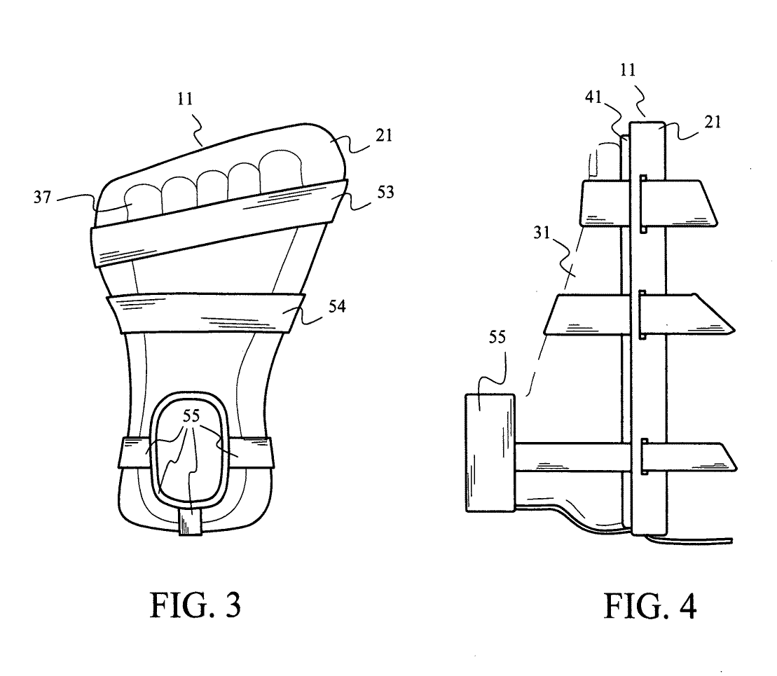

In the alternative, FIGS. 3 and 4 illustrate an exemplary design for a therapeutic device 11 adapted for a patient's foot 31. The therapeutic device 11 includes a contoured pad 21. The contoured pad 21 may be shaped along the outer silhouette of a foot 31 and/or any shape capable of restraining a portion of the patient's foot.

The contoured pad 21 is also made of a solid material to brace the foot 31. The contoured pad 21 may include any number of solid materials suitable for providing a rigid structure in order to provide a sufficient force to overcome the deformed foot of a person who has suffered from a severe accident, such as where a person has suffered a stroke and/or suffered from a lack of oxygen which resulted in a severe muscular deformity. For example, a severe loss of muscular control may result in the flat lower portion of a person's foot curling downward such that the toes curl downward toward the heel of the foot into an unsightly and unnaturally hyper-flexed clenched position.

The surface of the contoured pad 21 that the foot 31 is braced against may also be supported by a flat soft cushion 41 as described above. The cushion 41 may be disposed on the contoured pad 21 in a variety of ways as described above with respect to the hand 30. The cushion 41 and/or the contoured pad 21 may also include a depression 37 that the patient's foot 31 can be secured into to provide added comfort and to accurately position the toes of the patient's foot 31 in a desired orientation. In at least another aspect of the configuration of the therapeutic device 10, less emphasis is placed on the back side of the contoured pad 21 as it may be primarily used for patients who are unconscious and will not be applying any pressure onto any other object.

Straps 50, 53, 54, 55 are disposed directly on a surface of the contoured pad 21 and adjacent to the person's foot 31. As shown, a first strap 53 is tightened down to overcome the deformity and fastens the patient's toes onto the contoured pad 21. A second strap 54 is shown securing the patient's forefoot to the contoured pad 21. In addition, straps 55, 56 are provided to overcome the gross curling effect of the injured foot and to secure the ankle and heel of the foot 31 to the contoured pad 21. Length, width and placement of the various straps 50, 53, 54, 55 about the severely hyper-flexed deformed foot is critical. The straps 50 and the contoured pad 20 are used together to simultaneously provide sufficient strength to overcome the hyper-flexed deformed foot as described above in the unnaturally deformed state in which atrophy of the muscle may have set in and/or the patient is unconscious (and consequently cannot voluntarily place any of his weight onto his foot) and/or is unwilling to assist in overcoming the severe deformity.

As mentioned above, the straps 50, 53, 54, 55 may include and/or be substituted by any number of mechanisms for securing, and any number of straps may be applied to the upper surface of the contoured pad 20 to hold the patients foot 31 in a desired orientation. Numerous straps can also be provided to secure each of the toes individually in place. Alternatively, a single strap can be interwoven within the contoured pad 21 or the cushion 41 to create many loops to receive each of the patient's toes individually.

Therapeutic Heat Stimulation

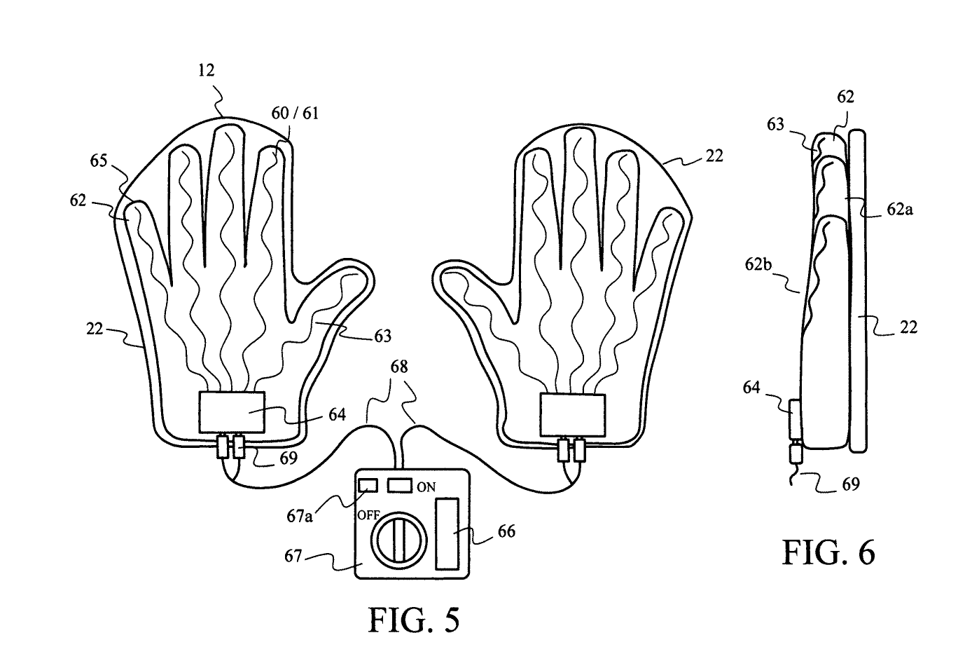

FIGS. 5-6 illustrate a therapeutic device 12 according to this invention that includes a therapeutic stimulation mechanism 60. The stimulation mechanism 60 includes a heating mechanism 61. The heating mechanism 61 includes a glove 62 that is worn on the hand 30 of a patient. The glove 62 is attached to a rigid contoured pad 22. Although the contoured pad 22 is shown disposed adjacent to the glove 62, the contoured pad 22 may be integrated within the glove 62. The rigid contoured pad 22 may be attached to either the top or bottom side of the glove 62.

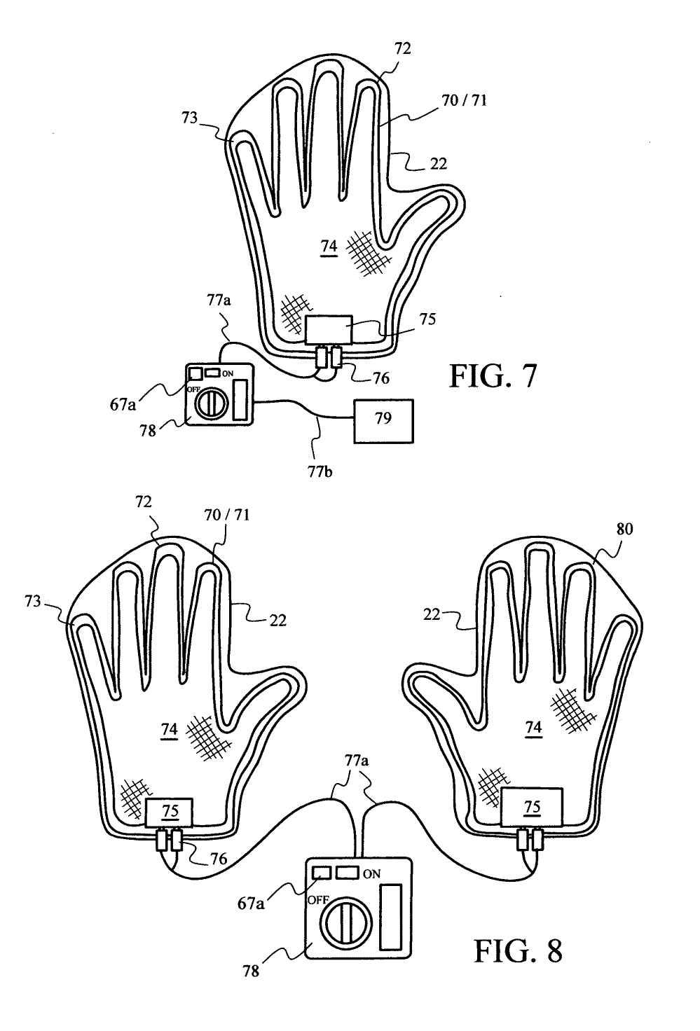

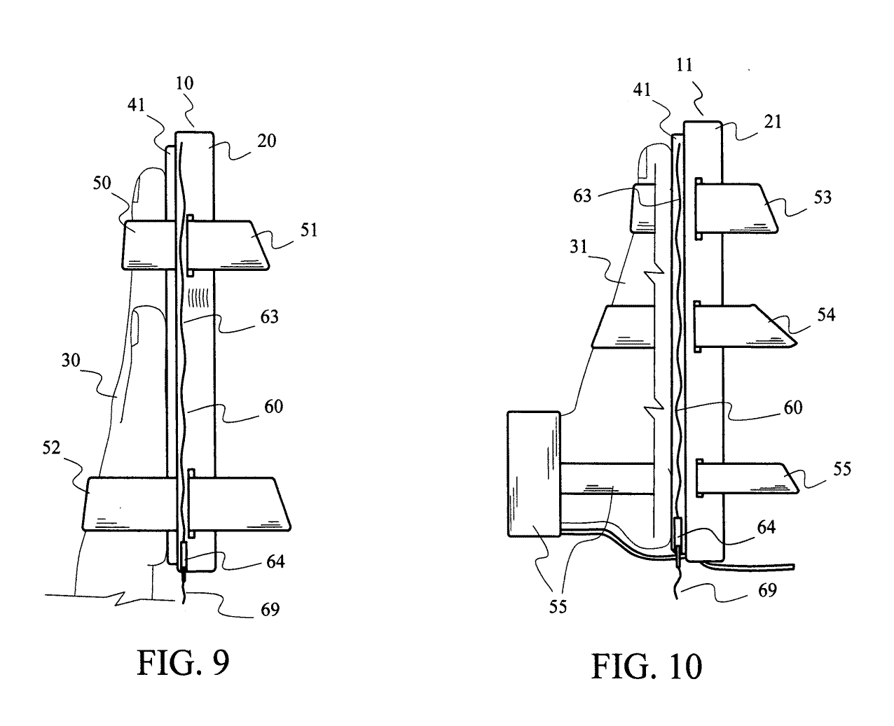

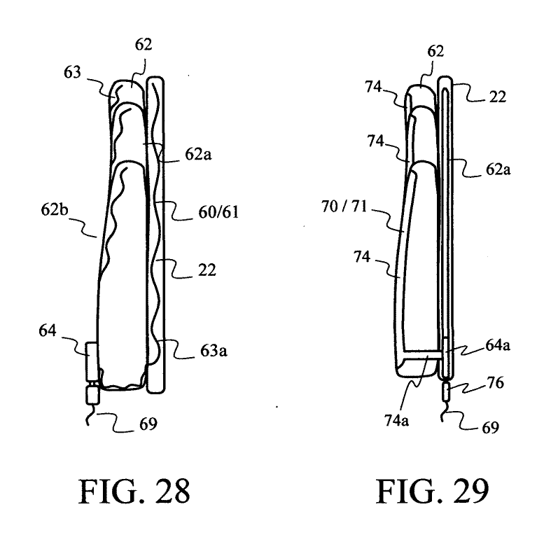

Circuitry of the heating mechanism 61 can be integrated into the contoured pad 22. FIGS. 9 and 28 demonstrate the heating therapeutic mechanism 60 disposed within the contoured pad 20. Alternatively, the heating mechanism 61 may be embedded in the glove 62 (as shown in FIGS. 6 and 28). The heating mechanism 61 may also be disposed within the cushion 41 (as shown in FIG. 10) without departing from the scope of the invention. In the alternative, the heating mechanism 61 may be integrated into the straps 50, 51-56 (as will be discussed later in more detail) and/or any other fasteners provided to secure the hand 30 or foot 31 of the patient to the contoured pad. Similar construction applies for the electrical stimulation mechanism, as will be described in more detail below with respect to FIGS. 7 and 8.

Referring to FIG. 6, the glove 62 includes a bottom portion 62a that covers the palm side of the hand 30 and top portion 62b that covers the back side of the hand 30. The glove may be manufactured from a variety of materials, including a stretchable fabric, such as LYCRA, cotton, a polymer and or any other suitable material which closely conform to the shape of the patient's hand 30 that is adapted to support the heating mechanism 61 and/or the electrical therapeutic stimulation mechanism 70 as will be described in more detail later.

As shown in FIG. 5, the heating mechanism 61 includes various thin electrodes and/or heat conductive wires 63 that extend into each of the fingers 65 in the glove 62 from a plug portion 64 disposed in the glove 62 adjacent to the wrist portion of the glove 62. The plug portion 64 may be embedded anywhere in the fabric of the glove 62 and is not limited to the wrist portion.

The heating mechanism 61 includes a power supply 66 attached to a heating control portion 67. The heating control portion 67 is electrically connected to the plug portion 64. The heating control portion 67 can be remotely controlled from the glove 67. Alternatively, the heating control portion 67 may be disposed on the glove and controlled at the glove 62 such that heating mechanism 61 could be a completely self-sustained heating unit. The heating mechanism 61 can be composed of a variety of methods now known or later described which operably warms the patient's hand 30 within the glove 62. For example, the glove may be comprised of a metal coated fabric inside or outside of the glove and may be connected from a wire 68 to a heating control portion 67 through the plug portion 64.

The power supply 66 may be disposed in a battery compartment of the heating control portion 67. The heating control portion 67 may be configured to have terminals connected through wires 68 to a socket 69 that mates with the plug portion 64.

In operation, electrical power is provided from power supply 66 through socket 69 to plug portion 64 to wires 63 in the glove 62 when the stimulation mechanism 60 is powered on. The amount of heat generated in the glove 62 can be controlled in a variety of known methods, for example, by modulation of the width of the drive pulses as a function of temperature. A processor 67a (as described in more detail later) may be incorporated for programmable control.

The power supply 66 may be contemplated from any number of different power sources, for example, the power source could be solar powered or some other type of battery. The power source can also be mounted in a number of other locations in accordance with this invention.

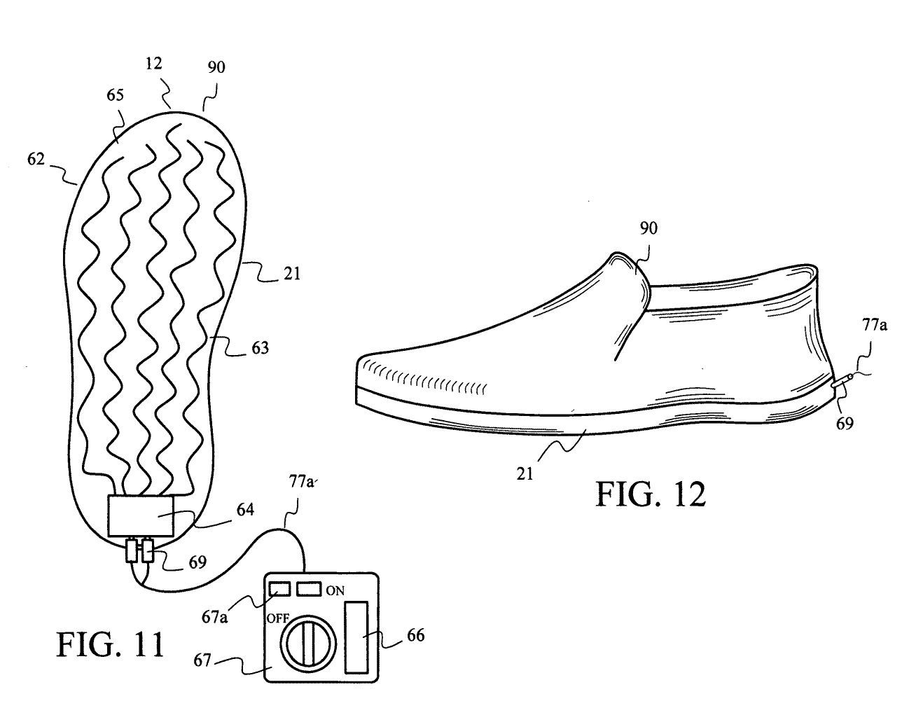

In the alternative, the stimulation mechanism 60 including the heating mechanism 61 can be incorporated into a sock-like booty 90 for the patient's foot 31 as shown in FIGS. 11 and 12 attached to the contoured pad 21. The heating mechanism 61 would operate similarly to the mechanism integrated for the patient's foot 31 as shown in FIG. 5.

As briefly mentioned above, the heating mechanism 61 may be disposed within the various straps 50-56. The advantage being that the therapeutic stimulation from the therapeutic device may be provided over the top of the foot as well as from below and from the sides of the foot. The even distribution of the therapeutic stimulus applied to the foot (and/or hand) fosters a more efficient recovery and more thorough rehabilitation to the extremity.

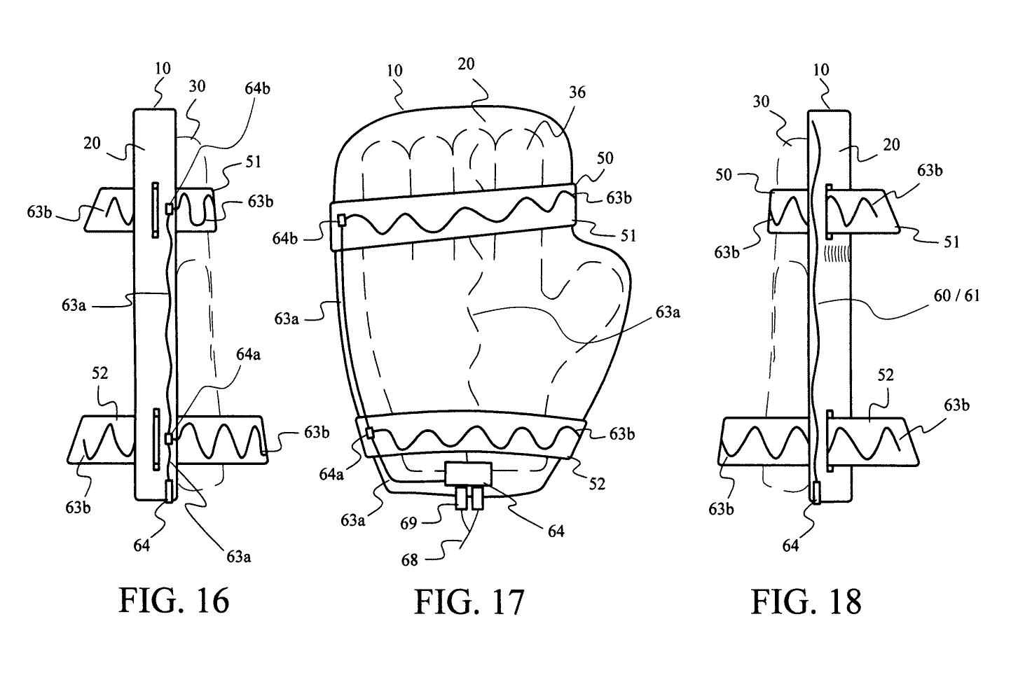

FIGS. 16-18 demonstrate an exemplary embodiment in which the various straps 50-52 of the therapeutic device 10 include heat conductive wiring 63b. That is, the plug portion 64 is connected to a first conductive wire 63a which extends from the plug portion 64 to a first conductive junction 64a. At the junction 64a, a first wire 63b extends from the first conductive junction 64a across the strap 52 which is adapted to secure the wrist of the user, as well as to provide therapeutic heating over the top portion of the wrist portion of the person as well as completely around the hand (or foot) in an evenly distributed manner thereby assisting in the rehabilitation of that extremity.

A second conductive wire 63a extends from the first conductive junction 64a to a second conductive junction 64b. At the second junction 64b, a second heat conductive wire 63b extends from the second conductive junction 64b across the strap 51 which is adapted to secure the fingers of the user to the rigid contoured pad 20. Simultaneously, the second heat conductive wire 63b provides therapeutic heating over the top, and sides of the finger portion of the hand of the person.

Likewise, as described above with respect to FIGS. 9-10, the heating mechanism 61 is shown in FIGS. 16-18 integrated into the contoured pad 20. According to this exemplary embodiment, therapeutic heat stimulation may be provided more uniformly to the palm of the hand as well as simultaneously to the top of the hand of the user.

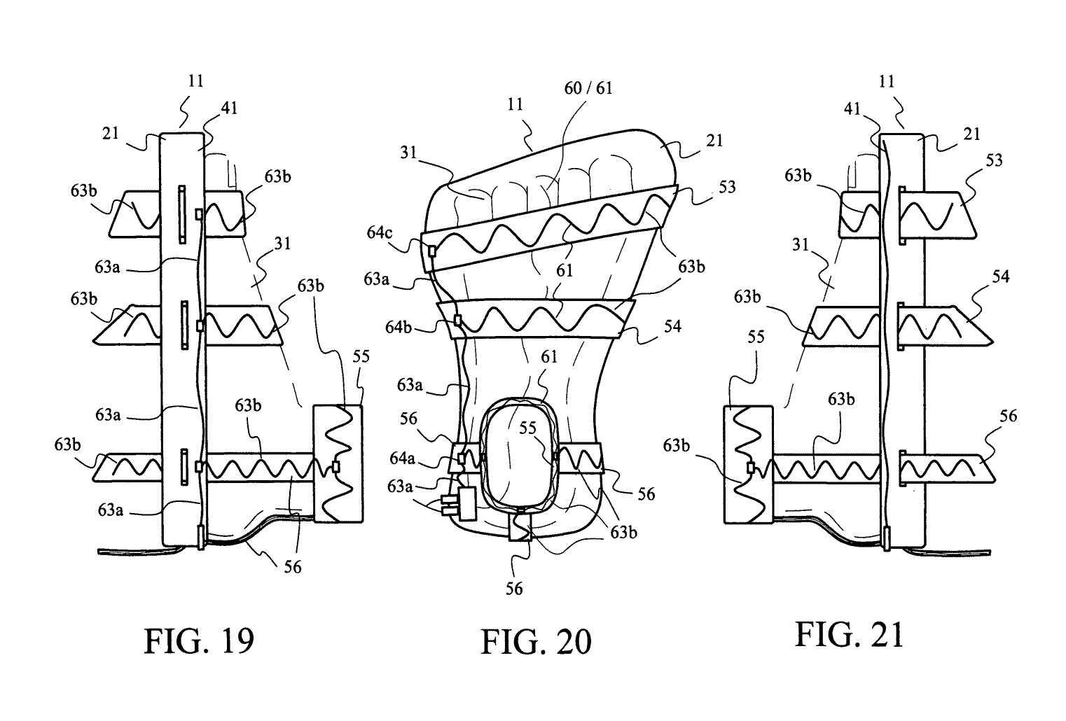

FIGS. 19-21 further demonstrate the heating mechanism 61 being disposed within the various straps 50-56 of the therapeutic device 11 as well as within the contoured pad 21. That is, the various straps 50-52 of the therapeutic device 11 include various conductors and/or heat conductive wires 63b that makes up the therapeutic device 11. As before, the plug portion 64 is connected to a first conductive wire 63a which extends from the plug portion 64 to a first conductive junction 64a. At the first junction 64a, a first wire 63b extends from the first conductive junction 64a through a series of straps 56 and an ankle encircling strap 55 which is adapted to firmly secure the ankle of the user, as well as to provide therapeutic heating around the ankle portion of the person.

A second conductive wire 63a extends from the first conductive junction 64a to a second conductive junction 64b. At the second junction 64b, a second heat conductive wire 63b extends from the second conductive junction 64b across the strap 54 which is adapted to secure the forefoot of the user securely to the rigid contoured pad 21. Simultaneously, the second heat conductive wire 63b provides therapeutic heating over the top of the forefoot of the person.

A third conductive wire 63a extends from the second conductive junction 64b to a third conductive junction 64c. At the third junction 64c, a third heat conductive wire 63b extends from the third conductive junction 64c across the strap 53 which is adapted to secure the toe portion of the user securely to the rigid contoured pad 21. Simultaneously, the third heat conductive wire 63b also provides therapeutic heating over the top of the toes of the person.

Likewise, as described above with respect to FIGS. 9-10, the heating mechanism 61 is shown in FIGS. 19-21 integrated into the contoured pad 20. According to this exemplary embodiment, therapeutic heat stimulation may be provided more uniformly to the top of the foot as well as, simultaneously, to the sides and bottom of the foot of the user. Enhanced therapy to the traumatized foot (or hand) can be more efficiently obtained according to this exemplary embodiment through the even distribution of therapy around the various sides of the extremity.

Therapeutic Electrical Stimulation

FIGS. 7 and 8 illustrate another exemplary embodiment of the invention. The therapeutic stimulation mechanism 70 includes an electrical mechanism 71 that provides a therapeutic electro-massage to stimulate the patient's hand 30 or foot 31. The electrical mechanism 71 can be adapted to function in a glove 72 that is worn on the hand 30 of a patient as shown in FIG. 7. In the alternative, the circuitry of the electrical mechanism 71 can be integrated into a rigid contoured pad 22 secured to the glove 72 to operate in conjunction with the glove 72 or straps 50 where the straps serve as electrodes whose functionality will be shown and described below.

FIG. 7 illustrates an electrode glove 72 according to this invention. The electrode glove 72 includes an outer insulating portion 73 formed of a flexible elastomeric material. The elastomeric material may be composed of a variety of different insulating materials suitable for providing flexibility and insulation in accordance with this invention, such as latex rubber and the like. The electrode 74 may be a thin layer of electrically conductive but nonmetallic, flexible material may be disposed on all palm and finger surfaces of the glove 72.

Adjacent to the wrist portion of the glove 72, a connector 75 portion may be disposed of the same material as the electrode 74 and is integral with the electrode 74. The connector 75 portion may include one or more receiving apertures 76 adapted to accept lead wires 77a, 77b of a transcutaneous electrical nerve stimulation (TENS) unit 78 for the purpose of applying a combination of TENS and whole-hand massage (electro-massage) or acupuncture (electro-acupuncture) to a patient to reduce the pain to a patient's hand 30 or foot 31.

The electrode 74 may be fabricated from a thin layer of an elastomeric material such as carbonized rubber or carbonized silicone. The electrode 50 may be applied to the outer insulating portion 73 with an adhesive and/or it may be deposited as a coating.

TENS units are known in the art. According to this invention, the TENS unit 78 may be configured as a small, portable, battery operated device which emits biphasic pulses having zero net direct current. The amplitudes of the pulses may be up to sixty milliamps with durations of up to 500 microseconds and pulse rates of up to 200 hertz. The current emitted by TENS units which are powered by higher voltages, for example 220 volt AC, can be much greater than those previously described. For example, one type of AC powered TENS unit known as a high voltage pulsed galvanic stimulator (HVPGS), is capable of emitting pulses of up to 500 volts. Another type of TENS unit known as the Russian Faradic Unit, can emit pulses at rates up to 2000 hertz. A special class of TENS unit known as a Functional Electrical Stimulation (FES) unit, is a battery operated unit which is specifically designed to elicit timed cyclical contractions to strengthen muscles or prevent atrophy from disuse.

The electrode glove 72 according to the present invention provides for a method of combining the therapeutic effects of TENS with the benefit of manual massage when the glove is connected to a TENS, FES, or HVPGS unit.

In operation, one lead wire 77a from a TENS unit output channel is connected to energize the glove electrode 74, while the other lead wire 77b may be connected to energize a large indifferent electrode 79 attached to the patient. According to another aspect of this invention, as shown in FIG. 8, a second electrode glove 80 may be worn on the patient's other hand. The first glove 72 may be connected to the anode of a TENS unit output channel and the second glove 80 may be connected to the cathode of the channel. If more power than is normally available from a single channel of a TENS unit is needed, the lead wires 87 of similar polarity from each channel of a dual channel unit which has output channels that fire synchronously may be connected to a first glove electrode and the remaining lead wires of similar polarity may be connected to the second glove electrode or an indifferent electrode.

According to this configuration, a therapist or the patient can apply a deep electro-massage to substantially the entire hand. The stimulating electrodes can be significantly separated from each other to permit maximum current penetration and prevent shorting between the electrodes. Increases in current density are minimized which can result from small point type electrodes used in known devices.

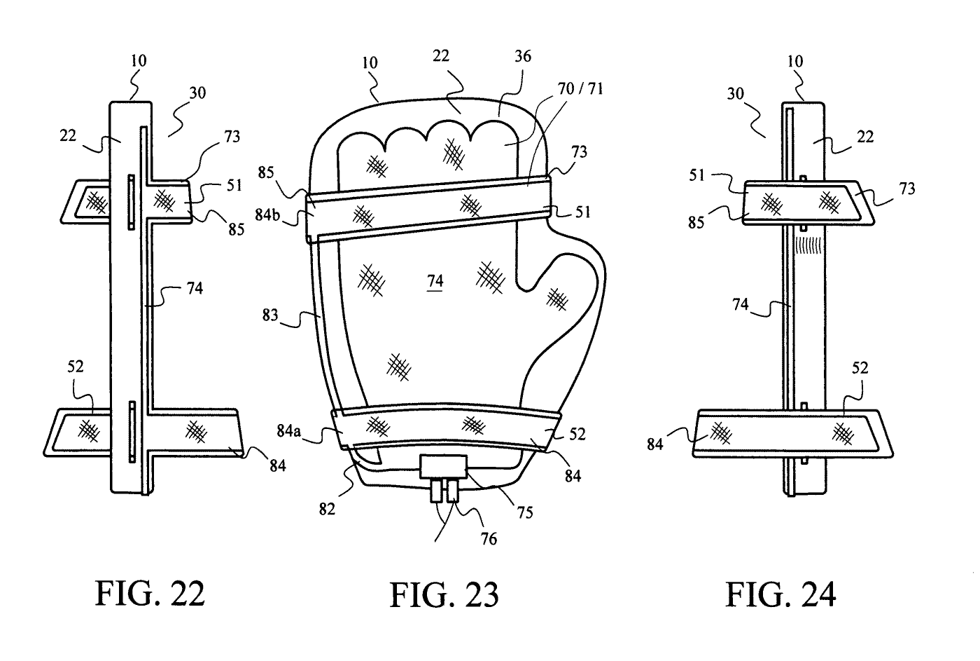

As mentioned briefly above, the circuitry of the electrical mechanism 71 can be integrated into a rigid contoured pad 22 where the various straps 50-56 serve as electrodes for the therapeutic stimulation mechanism. FIGS. 22-24 depict an exemplary embodiment in which the various straps 50-52 of the therapeutic device 10 includes various electrodes 74, 82-85 disposed throughout the therapeutic stimulation mechanism.

FIGS. 22-24 illustrates the connector 75 being electrically connected to a first electrode 74 disposed near the surface of the rigid contoured pad 22 as shown resembling the contour of a hand. The connector 75 is also electrically connected through a smaller electrode 82 to a first electrode junction 84a. At the electrode junction 84a, a first electrode 84 extends from the first electrode junction 84a through the strap 52 which is adapted to secure the wrist of the user, as well as to provide electric therapeutic stimulation over the top of the wrist portion of the person.

At the electrode junction 84a, another smaller first electrode 83 extends from the first electrode junction Ma to a second electrode junction 84b. At the second electrode junction 84b, a second electrode 85 extends from the second electrode junction 84b through the strap 51, which is adapted to secure the fingers of the user, as well as to provide electric therapeutic stimulation over the top of the fingers of the person.

Each of the straps 51, 52 (and 52-55) includes an outer insulating portion 73 formed of a flexible elastomeric material. The elastomeric material may be composed of a variety of different insulating materials suitable for providing flexibility and insulation, such as latex rubber and the like. The various electrodes 74, 82-85 may be a thin layer of electrically conductive but nonmetallic, flexible material.

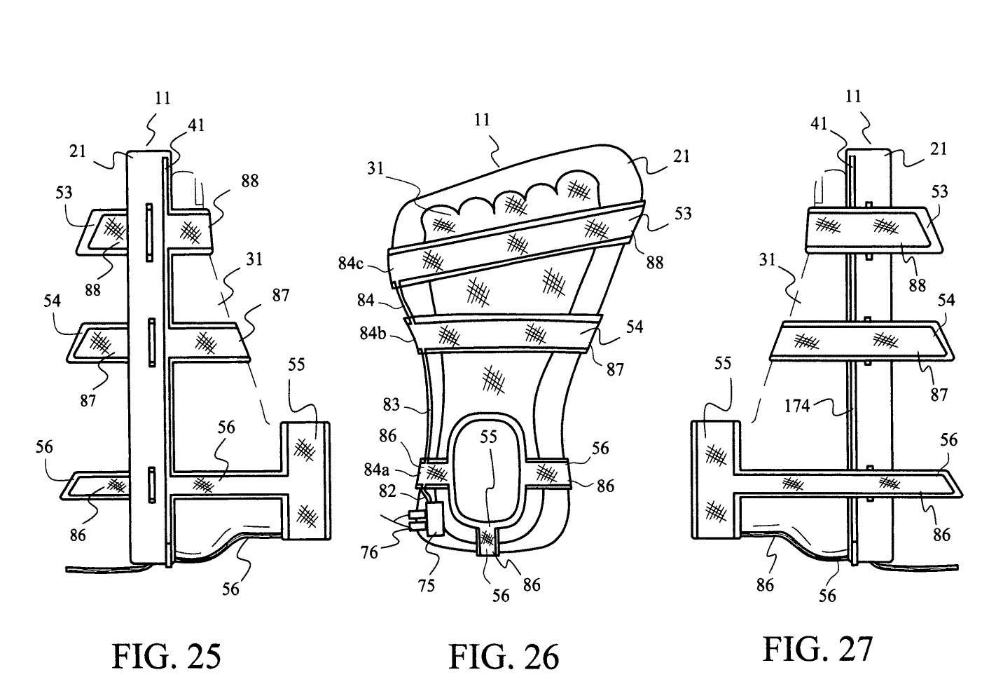

FIGS. 25-27 further depict the circuitry of the electrical mechanism 71 being integrated into a rigid contoured pad 21 of a therapeutic device 11 where the various straps 50-56 serve as electrodes for the therapeutic stimulation mechanism. That is, FIGS. 25-27 illustrate an exemplary embodiment in which the various straps 50-52 of the therapeutic device 11 includes various electrodes 74, 82-88 disposed throughout the therapeutic stimulation mechanism.

FIGS. 25-27 illustrates the connector 75 being electrically connected to a first electrode 174 disposed near the surface of the rigid contoured pad 21 as shown resembling the contour of a foot. The connector 75 is electrically connected through a smaller electrode 82 to a first electrode junction 84a. At the first electrode junction 84a, a first electrode 84 extends from the first electrode junction 84a through a series of straps 56 and an ankle encircling strap 55 which is adapted to firmly secure the ankle of the user, as well as to provide therapeutic electric stimulation around the ankle portion of the person in an evenly distributed manner thereby assisting in the rehabilitation of that extremity.

A second smaller electrode 83 extends from the first conductive junction 84a to a second conductive junction 84b. At the second junction 84b, a second electrode 87 extends from the second electrode junction 84b through the strap 54 which is adapted to secure the forefoot of the user, as well as to provide electric therapeutic stimulation over the top of the forefoot portion of the person.

From the second junction 84b, the a third smaller electrode 84 extends from the second electrode junction 84b to a third electrode junction 84c. At the third electrode junction 84c, a third electrode 88 extends from the third electrode junction 84c through the strap 53, which is adapted to secure the toes of the user, as well as to provide electric therapeutic stimulation over the top of the toes of the person in an evenly distributed manner thereby assisting in the rehabilitation of that extremity.

The combined use of electrodes 82-88 in the straps of the therapeutic mechanism 11 above the user's foot, as well as the use of electrode 174 integrated into the contoured pad 20 provides greater uniformity to the bottom, the side and the top of the foot simultaneously in an evenly distributed manner. Quicker recovery and enhanced therapeutic results to the traumatized foot can be obtained according to this exemplary embodiment.

Therapeutic Device Combined with Heat and Electrical Stimulation

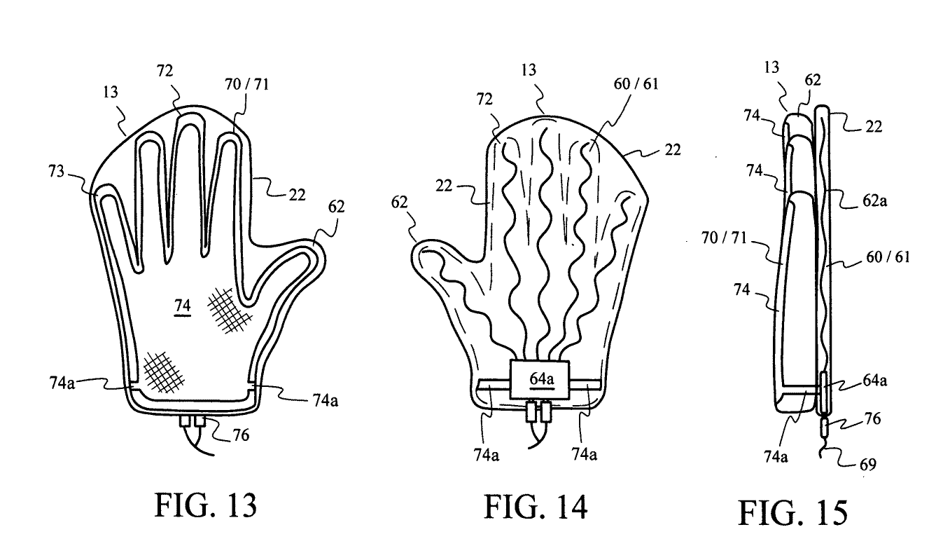

FIGS. 13, 14 and 15 show a therapeutic device 13 configured as a glove 62 operably integrating both an electrical mechanism 71 and a heating mechanism 61 into a single therapeutic stimulation system, such as the glove 72 illustrated. According to this embodiment, both heating and electrical stimulation is possible individually and/or simultaneously. Although shown as a glove 72, the therapeutic device 10 may be configured as a sock-like booty (similar to the embodiment shown is FIGS. 11 and 12) that operably integrates the electrical mechanism 71 and the heating mechanism 61 into a single therapeutic stimulation system so that both heating and electrical stimulation is also possible individually and/or simultaneously.

FIGS. 13-15 illustrate an electrode glove 72 including an electrode 74 surrounded by an outer insulating portion 73. The electrode 74 is connected to a plug portion 64a disposed adjacent to the heating mechanism 61 through electrode lead straps 74a that extend from the electrode 74 to the plug portion 64a. The plug portion 64a according to this embodiment is adapted to provide connectivity for both the electrical mechanism 71 and the heating mechanism 61 as a single therapeutic stimulation mechanism. The operative function for the electrical mechanism 71 and the heating mechanism 61 according to this embodiment function as described before but may be programmed to act separately, or in conjunction with each other, as desired by the user or person prescribing a particular mode of therapy.

As is known in the art, the electrical mechanism 71 and the heating mechanism 61 would be isolated from each other to prevent interference of their separate functions. Alternatively, the electrical mechanism 71 can be modified to provide heat (as well as to provide electrical pulse stimulation) as a heating mechanism to predetermined regions of the patient's hand 30 or foot 31.

According to this invention, a processor 67a may be implemented at various elements of this therapeutic device 10. The processor 67a may be implemented in association with, and/or as part of, the heating control portion 67, the electrical mechanism 71, the connectors 64, 75, and/or in association with any portion of the therapeutic device 10. The processor 67a may be implemented as a programmed general purpose computer, on a special purpose computer, a programmed microprocessor or micro-controller and peripheral integrated circuit elements, an ASIC or other integrated circuit, a digital signal processor, a hardwired electronic or logic circuit such as a discrete element circuit, a programmable logic device such as a PLD, PLA, FPGA or PAL, or the like. In general, any device processor capable of implementing the therapeutic functions shown in the figures can be used according to this invention. The particular form taken for the processor 67a is a design choice and will be obvious and predicable to those skilled in the art.

The processor 67a of the therapeutic device 10 can be associated with a storage unit (not shown) using any appropriate combination of alterable, volatile or non-volatile memory or non-alterable, or fixed, memory. The alterable memory, whether volatile or non-volatile, can be implemented using any one or more of static or dynamic RAM, a floppy disk and disk drive, a write-able or rewrite-able optical disk and disk drive, a hard drive, flash memory or the like. Similarly, the non-alterable or fixed memory can be implemented using any one or more of ROM, PROM, EPROM, EEPROM, an optical ROM disk, such as a CD-ROM or DVD-ROM disk, and disk drive or the like.

The processor 67a of the therapeutic device 10 is programmable and can be adapted to provide instruction to the therapeutic stimulation mechanism 60, 70. As mentioned previously, the therapeutic stimulation mechanism can include any one of, or both, the electrical stimulation mechanism 70 and/or the heating stimulation mechanism 60 in a single embodiment according to this invention. The processor 67a is programmable to provide predetermined amounts of heating and/or electrical simulation to the preferred body portion.

It will be recognized by those skilled in the art that changes or modifications may be made to the above described embodiment without departing from the broad inventive concepts of the invention. It is understood therefore that the invention is not limited to the particular embodiment which is described, but is intended to cover all modifications and changes within the scope and spirit of the invention.

What is claimed is:

1. A therapeutic device for biasing a body portion of a patient into a natural unclenched predetermined position, comprising:
a rigid support that is substantially flat and contoured to brace the body portion into the natural unclenched predetermined position;
a securing mechanism attached directly to the rigid support that firmly braces, and biases, an unnaturally clenched body portion completely back into the natural unclenched predetermined position against the rigid support; and
a therapeutic stimulation mechanism associated with the rigid support that is adapted to provide stimulation to the body portion.

2. The therapeutic device recited in claim 1, wherein the rigid support includes a cushion secured to the rigid support.

3. The therapeutic device recited in claim 1, wherein the rigid support includes a depression to align the body portion in a preferred orientation.

4. The therapeutic device recited in claim 1, wherein the body portion is a foot.

5. The therapeutic device recited in claim 1, wherein the securing mechanism is a sock-like booty fastened to the surface of the rigid support.

6. The therapeutic device recited in claim 1, wherein the securing mechanism includes at least one of a strap, a buckle, a Velcro fastener, a clamp, and a tie.

7. The therapeutic device recited in claim 1, wherein the therapeutic stimulation mechanism is a heating device that provides heat stimulation to the body portion.

8. The therapeutic device recited in claim 1, wherein the therapeutic stimulation mechanism is integrated as part of the rigid support.

9. The therapeutic device recited in claim 1, wherein the therapeutic stimulation mechanism is integrated as part of the securing mechanism.

10. The therapeutic device recited in claim 7, wherein the heating device includes a plug connector portion into which a power supply is connected and from which wires extend to provide warmth along a length of the body portion by a heating control unit.

11. The therapeutic device recited in claim 7, wherein the heating device includes a programmable heating control unit that remotely controls warmth to the therapeutic device.

12. The therapeutic device recited in claim 7, wherein the heating device includes a programmable heating control unit that is controlled on the therapeutic device as a self-sustained therapeutic device.

13. The therapeutic device recited in claim 1, wherein the therapeutic stimulation mechanism is an electrical stimulation device to provide stimulation to the body portion.

14. The therapeutic device recited in claim 13, wherein the electrical stimulation device includes an outer insulating portion and a thin layer of electrically conductive material disposed on an upper surface of the securing mechanism where the body portion is the foot and the securing mechanism is a sock-like booty.

15. The therapeutic device recited in claim 13, wherein the therapeutic stimulation mechanism includes a connector portion including a transcutaneous electrical nerve stimulation (TENS) unit that applies at least one of: a TENS, whole-hand massage unit (electro-massage), and an acupuncture unit (electro-acupuncture) to the patient to reduce the pain to the body portion being unnaturally clenched.

16. A therapeutic device for biasing a hand of a patient into a naturally flat predetermined position, comprising:
a rigid support that is substantially flat and contoured to brace the hand into the naturally flat predetermined position;
a securing glove attached directly to the rigid support that firmly braces, and biases, an unnaturally deformed hyper-flexed hand completely back into the naturally flat predetermined position against the rigid support; and
a therapeutic stimulation mechanism associated with the rigid support that is adapted to provide stimulation to the hand.

17. The therapeutic device recited in claim 16, wherein the therapeutic stimulation mechanism is an electrical stimulation device that includes an outer insulating portion and a thin layer of electrically conductive material disposed on all palm and finger surfaces of the securing glove.
